(19) United States
(12) Patent Application Publication
(10) Pub. No.: US 2025/0223532 A1
(43) Pub. Date: Jul. 10, 2025

(54) SMART CELL CULTURE FLASK

(71) Applicant: BIOGREAT CO., LTD., Taoyuan City (TW)

(72) Inventor: Bing-Long HO, Taoyuan City (TW)

(21) Appl. No.: 19/001,788

(22) Filed: Dec. 26, 2024

(30) Foreign Application Priority Data
Jan. 4, 2024 (TW) 113200143

(51) Int. Cl. C12M 1/24; C12M 1/00; C12M 1/34

(52) U.S. Cl. CPC C12M 23/08; C12M 23/52; C12M 41/30

(57) ABSTRACT

A smart cell culture flask of the present invention is used for containing a cell suspension and electrically connected to a blockchain network. The smart cell culture flask includes a culture flask main body, a chipset and a detection module. The culture flask main body is used for containing the cell suspension. The chipset is located in the culture flask main body, the chipset includes a blockchain information tracer, the blockchain information tracer is electrically connected to the blockchain network, and used for recording a source of the cell suspension and a logistics process of the smart cell culture flask on the blockchain network for traceability. The detection module is located in the culture flask main body and electrically connected to the chipset, wherein the detection module is used for detecting at least one cell suspension property of the cell suspension.

### CROSS REFERENCE TO RELATED APPLICATIONS

This application claims priority of application Ser. No. 11/320,0143 filed in Taiwan on Jan. 4, 2024 under 35 U.S.C. § 119, the entire contents of both of which are hereby incorporated by reference.

### BACKGROUND OF THE INVENTION

### 1. Field of the Invention

The present invention relates to a smart cell culture flask, more particularly, the present invention relates to a smart cell culture flask which is easy to trace the source of the cell suspension and detect the property of the cell suspension.

### 2. Description of the Related Art

A culture flask is a transparent glass or plastic bottle into which scientists pour a cell suspension containing microorganisms, such as cells or biological tissues, to cultivate microorganisms within the flask.

However, current culture flasks can only be used to cultivate the microorganisms in the cell suspension and are not capable of detecting the properties of the cell suspension; thus, the scientists must spend significant time constantly monitoring the properties of the cell suspension. Furthermore, current culture flasks cannot trace the source of the cell suspension. Therefore, how to provide a culture flask that can trace the source of the cell suspension and detect its properties, has become a topic worth exploring.

### SUMMARY OF THE INVENTION

The main object of the present invention is to provide a smart cell culture flask which is easy to trace the source of the cell suspension and detect the property of the cell suspension.

To achieve the abovementioned object, a smart cell culture flask of the present invention is used for containing a cell suspension and electrically connected to a blockchain network. The smart cell culture flask includes a culture flask main body, a chipset and a detection module. The culture flask main body is used for containing the cell suspension. The chipset is located in the culture flask main body, the chipset includes a blockchain information tracer, the blockchain information tracer is electrically connected to the blockchain network, and used for recording a source of the cell suspension and a logistics process of the smart cell culture flask on the blockchain network for traceability. The detection module is located in the culture flask main body and electrically connected to the chipset, wherein the detection module is used for detecting at least one cell suspension property of the cell suspension.

According to one embodiment of the present invention, wherein the chipset further includes a positioning unit, and the positioning unit is used for positioning the smart cell culture flask.

According to one embodiment of the present invention, wherein the chipset further includes a temperature detector, and the temperature detector is used for detecting a temperature of the smart cell culture flask.

According to one embodiment of the present invention, wherein the chipset further includes an electronic tag unit, and the electronic tag unit records an information of the smart cell culture flask via a Radio Frequency Identification method.

According to one embodiment of the present invention, the smart cell culture flask further includes a wireless module, wherein the wireless module is located in the culture flask main body and electrically connected to the chipset, the detection module and an external computer.

According to one embodiment of the present invention, the smart cell culture flask further includes a battery, wherein the battery is located in the culture flask main body and providing power to the chipset, the detection module and the wireless module.

According to one embodiment of the present invention, wherein the culture flask main body further includes a bottle body, and the chipset, the detection module, the wireless module and the battery are located in the bottle body.

According to one embodiment of the present invention, wherein the detection module is located on an inward side of the bottle body.

According to one embodiment of the present invention, wherein the positioning unit is a Global Positioning System chip.

### BRIEF DESCRIPTION OF THE DRAWINGS

FIG. 1 is a schematic diagram of a smart cell culture flask according to one embodiment of the present invention.

FIG. 2 is a schematic diagram of a smart cell culture flask which contains a cell suspension according to one embodiment of the present invention.

FIG. 3 is a system structure diagram of a smart cell culture flask, an external computer, an electronic tag reader, and a blockchain network according to one embodiment of the present invention.

### DETAILED DESCRIPTION OF THE PREFERRED EMBODIMENT

To provide a clearer understanding of the technical content of the present invention, a preferred embodiment is described as follows.

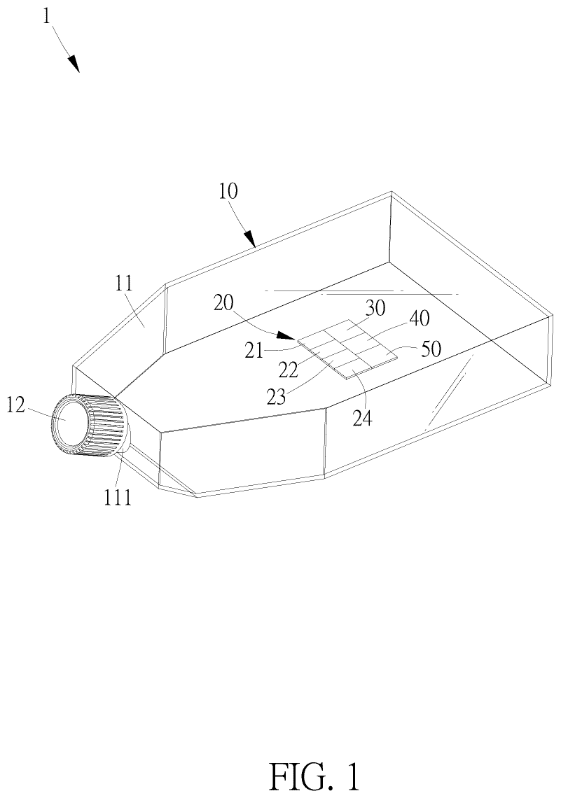

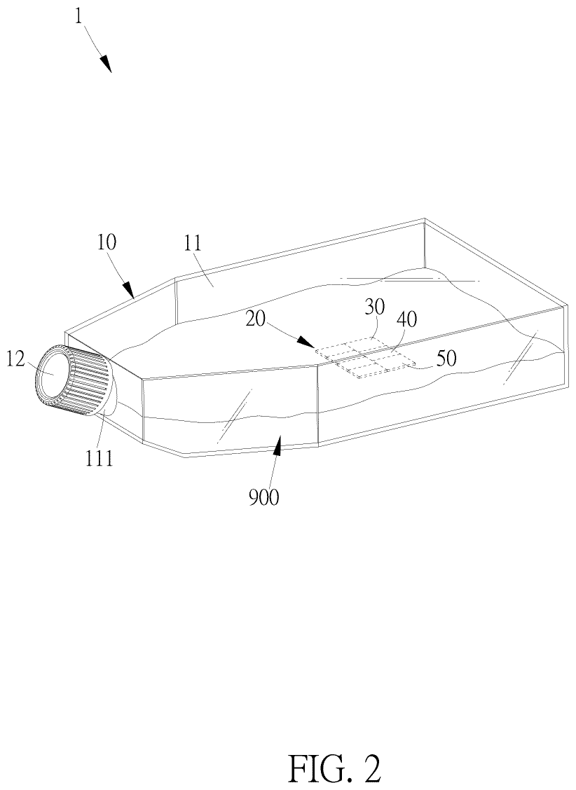

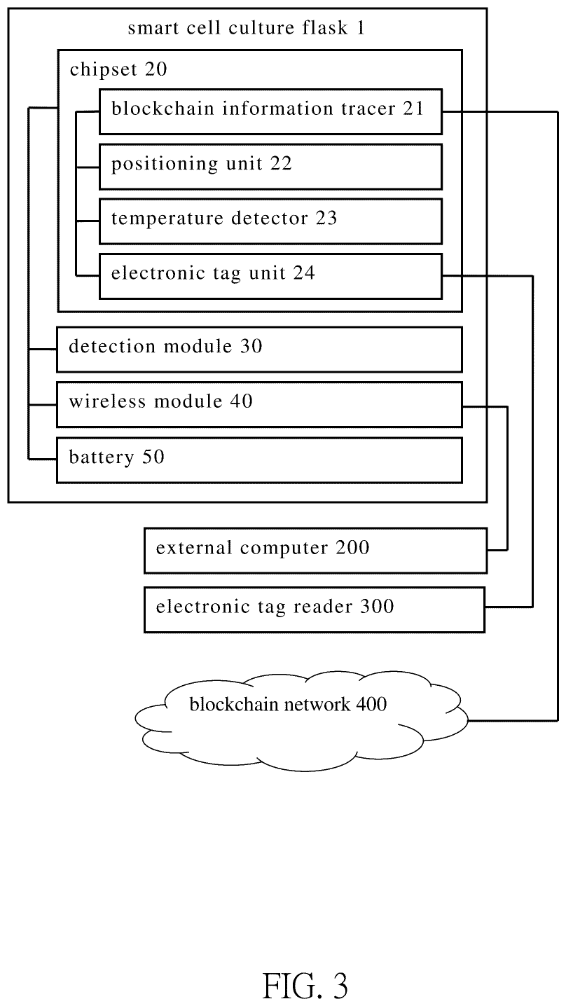

Please refer to FIG. 1 to FIG. 3, which illustrate the smart cell culture flask according to one embodiment of the present invention. FIG. 1 is a schematic diagram of a smart cell culture flask according to one embodiment of the present invention. FIG. 2 is a schematic diagram of a smart cell culture flask which contains a cell suspension according to one embodiment of the present invention. FIG. 3 is a system structure diagram of a smart cell culture flask, an external computer, an electronic tag reader, and a blockchain network according to one embodiment of the present invention.

As shown in FIG. 1 to FIG. 3, in one embodiment of the present invention, the smart cell culture flask 1 is easy to trace the source of the cell suspension and detect the cell suspension properties. The smart cell culture flask 1 is used for containing a cell suspension 900 and is electrically connected to an external computer 200 and a blockchain network 400, to transmit relevant information about the smart cell culture flask 1 and the contained cell suspension 900 to the external computer 200 and the blockchain network 400. The cell suspension 900 is a liquid containing microscopic cells, such as bacteria, viruses, fungi, algae, or biological cells, which are too small to be seen with the naked eye; however, the types of microscopic cells are not limited to those mentioned above and can be adjusted based on actual usage needs. The external computer 200, such as a desktop computer or laptop, is used for mutual data transmission with the smart cell culture flask 1. The blockchain network 400 is an external network system that uses blockchain technology to establish and store data chains.

In one embodiment of the present invention, the smart cell culture flask 1 includes a culture flask main body 10, a chipset 20, a detection module 30, a wireless module 40, and a battery 50. The culture flask main body 10 is made of glass or plastic and is used for containing a cell suspension 900. The culture flask main body 10 includes a bottle body 11 and a cover 12. The bottle body 11 is used by the staff to load the cell suspension 900. The chipset 20, the detection module 30, the wireless module 40, and the battery 50 are installed in the bottle body 11; the detection module 30 is located on the inward side of the bottle body 11. The bottle body 11 includes an opening 111 which is used to let the cell suspension 900 in and out. The cover 12 is used for sealing the opening 111, ensuring the bottle body 11 is airtight and preventing the cell suspension 900 from leaking out.

In one embodiment of the present invention, the chipset 20 is installed on the bottle body 11 of the culture flask main body 10. The chipset 20 includes a blockchain information tracer 21, a positioning unit 22, a temperature detector 23 and an electronic tag unit 24. The blockchain information tracer 21 is a processing chip capable of electrically connecting to a blockchain network 400, and transmits and records data in the blockchain network 400 and can also trace data previously recorded in the blockchain network 400. For example, the blockchain information tracer 21 records the source and the logistics process of the cell suspension 900, and the logistics process of the smart cell culture flask 1, into the blockchain network 400. Utilizing the traceability and tamper-resistant characteristics of blockchain, the system tracks and records the source and logistics process of the cell suspension 900 and the logistics process of the smart cell culture flask 1. The blockchain information tracer 21 can also trace the source and logistics process of the cell suspension 900 and the logistics process of smart cell culture flask 1 recorded on the blockchain network 400. According to one embodiment of the present invention, the source of the cell suspension 900 may be, for example, from a chemical laboratory or a pharmaceutical company. The logistics process of the cell suspension 900 includes the location where the cell suspension 900 was obtained and the transport process of the cell suspension 900. The logistics process of the smart cell culture flask 1 includes the locations where the smart cell culture flask 1 was manufactured and stored, and the transportation details of the smart cell culture flask 1. Additionally, the source and logistics process of the cell suspension 900, and the logistics process of the smart cell culture flask 1, can be input into the blockchain information tracer 21 via the external computer 200. The blockchain information tracer 21 can then record the source and logistics process of the cell suspension 900, and the logistics process of the smart cell culture flask 1 into the blockchain network 400. In this way, the traceability and tamper-resistant characteristics of blockchain ensure clear recording and management of the source and logistics processes of the cell suspension 900 and logistics processes of the smart cell culture flask 1. The positioning unit 22 is a Global Positioning System (GPS) chip that is used for positioning for the smart cell culture flask 1, to determine the current location of the smart cell culture flask 1. The temperature detector 23 is a chip with temperature-sensing functionality, designed to monitor the temperature of the smart cell culture flask 1. This allows for monitoring whether the smart cell culture flask 1 is maintained at an appropriate temperature, avoiding extreme heat or cold that could affect the quality of the cell suspension 900. The electronic tag unit 24 records information about the smart cell culture flask 1 via Radio Frequency Identification (RFID) technology. This information includes, for example, the manufacturing date of the smart cell culture flask 1, the date and location where the cell suspension 900 was cultured in the smart cell culture flask 1, the type of cell suspension 900, and other details. Information recorded by the electronic tag unit 24 can be input via the external computer 200. However, the recorded information of the smart cell culture flask 1 is not limited to the aforementioned data and can be adjusted based on design requirements. The information recorded by the electronic tag unit 24 can also be read wirelessly by an external electronic tag reader 300 using radio frequency signals, however, since electronic tag readers are widely used in the field of radio frequency technology and are not the focus of this invention, further details are omitted.

In one embodiment of the present invention, the detection module 30 is installed on the inward side of the bottle body 11 and is electrically connected to the chipset 20. The detection module 30 is a chip with the function of measuring cell suspension properties and is used to contact the cell suspension 900 to measure at least one property of the cell suspension 900. Examples of cell suspension properties include pH levels, viscosity, and bacterial concentration, among others. By utilizing the detection module 30's function of measuring the properties of the cell suspension, it is possible to determine whether the condition of the cell suspension 900 is optimal or degraded. However, the cell suspension properties that the detection module 30 can measure are not limited to the aforementioned examples and can be adjusted according to practical requirements.

In one embodiment of the present invention, the wireless module 40, for example, is a network chip. The wireless module 40 is installed in the bottle body 11 of the culture flask main body 10 and is electrically connected to the chipset 20, the detection module 30, and the external computer 200. The wireless module 40 is used for transmitting the information measured by the chipset 20 and the detection module 30 to the external computer 200, enabling staff to operate the external computer 200 to access the data of the cell suspension 900 contained in the smart cell culture flask 1. Such data may include the source and historical records of the cell suspension 900, the logistics process of the smart cell culture flask 1, and the cell suspension properties of the cell suspension 900.

In one embodiment of the present invention, the battery 50 is installed in the bottle body 11 of the culture flask main body 10 and provides power to the chipset 20, the detection module 30, and the wireless module 40, ensuring that the chipset 20, detection module 30, and wireless module 40 can operate properly.

As shown in FIG. 1 and FIG. 2, when the staff uses the smart cell culture flask 1 of the present invention to store the cell suspension 900, the staff can pour the cell suspension 900 into the bottle body 11. The detection module 30 will measure the properties of the cell suspension 900 within the bottle body 11, such as pH levels, viscosity, and bacterial concentration, among others. In this way, the condition of the cell suspension 900 can be confirmed as either optimal or degraded. Subsequently, the staff can seal the opening 111 of the bottle body 11 with the cover 12, thereby isolating the cell suspension 900 from the external environment. This prevents the cell suspension 900 from deteriorating and facilitates subsequent biological cultivation or testing.

Additionally, the positioning unit 22 is used for positioning the smart cell culture flask 1 and determining its current position, preventing the smart cell culture flask 1 from being lost. The temperature detector 23 is a chip with temperature sensing function, used for detecting the temperature of the smart cell culture flask 1. Via this function, it is possible to monitor whether the smart cell culture flask 1 is maintained at an appropriate temperature, avoiding overly hot or cold environments that could affect the quality of the cell suspension 900.

Additionally, the staff can operate the external computer 200 to access the wireless module 40 of the smart cell culture flask 1, to input information into the electronic tag unit 24, such as the manufacturing date of the smart cell culture flask 1, the date and location that the smart cell culture flask 1 was used to culture the cell suspension 900, the type of cell suspension 900, and other relevant details. This allows the staff to use the electronic tag reader 300 to quickly read the data recorded in the electronic tag unit 24 during subsequent culturing or testing for the cell suspension 900 in the smart cell culture flask 1, enabling quick access to related information about the smart cell culture flask 1 and the cell suspension 900.

Furthermore, the staff can operate the external computer 200 to input the source and logistics process of the cell suspension 900, and the logistics process of the smart cell culture flask 1, into the blockchain information tracer 21. The blockchain information tracer 21 then records the information on the blockchain network 400. Via the blockchain's function of tracing the logistics process of items, details such as the source of the cell suspension 900, the location where the cell suspension 900 was obtained, the location where the smart cell culture flask 1 was manufactured and stored, and the transportation process of the smart cell culture flask 1 can all be recorded on the blockchain network 400. In this way, via the blockchain's traceability and tamper-resistant characteristics, the source and logistics process of the cell suspension 900 and the logistics process of the smart cell culture flask 1 can be clearly recorded and managed. As a result, even if the cell suspension 900 is found to have issues (e.g., contamination or abnormalities), the staff can quickly trace the source and logistics process of the cell suspension 900 via the blockchain, identifying whether problem occurred in the source or logistics process that caused contamination or abnormalities.

Via the design of the smart cell culture flask 1 in the present invention, the smart cell culture flask 1 is equipped with diverse and convenient functionalities, which include tracing the source and logistics process of the cell suspension, determining the current location of the smart cell culture flask 1 via positioning, monitoring the temperature of the smart cell culture flask 1 to ensure it remains at an appropriate temperature, recording data related to the cell suspension and the smart cell culture flask 1 using Radio Frequency Identification (RFID) for quick access by staff, and measuring the cell suspension property of the cell suspension.

It should be noted that the above is merely an embodiment and is not limited to this embodiment. For example, any design that does not depart from the fundamental structure of the present invention shall fall within the scope of the claims of this patent, which should be interpreted based on the scope of the patent claims.

What is claimed is:

1. A smart cell culture flask, used for containing a cell suspension and electrically connected to a blockchain network, the smart cell culture flask comprising:
a culture flask main body, used for containing the cell suspension;
a chipset, located in the culture flask main body, wherein the chipset comprises a blockchain information tracer, the blockchain information tracer is electrically connected to the blockchain network, and used for recording a source of the cell suspension and a logistics process of the smart cell culture flask on the blockchain network for traceability; and
a detection module, located in the culture flask main body and electrically connected to the chipset, wherein the detection module is used for detecting at least one cell suspension property of the cell suspension.

2. The smart cell culture flask as claimed in claim 1, wherein the chipset further comprises a positioning unit, and the positioning unit is used for positioning the smart cell culture flask.

3. The smart cell culture flask as claimed in claim 2, wherein the chipset further comprises a temperature detector, and the temperature detector is used for detecting a temperature of the smart cell culture flask.

4. The smart cell culture flask as claimed in claim 3, wherein the chipset further comprises an electronic tag unit, and the electronic tag unit records an information of the smart cell culture flask via a Radio Frequency Identification method.

5. The smart cell culture flask as claimed in claim 4, further comprising a wireless module, wherein the wireless module is located in the culture flask main body and electrically connected to the chipset, the detection module and an external computer.

6. The smart cell culture flask as claimed in claim 5, further comprising a battery, wherein the battery is located in the culture flask main body and providing power to the chipset, the detection module and the wireless module.

7. The smart cell culture flask as claimed in claim 6, wherein the culture flask main body further comprises a bottle body, and the chipset, the detection module, the wireless module and the battery are located in the bottle body.

8. The smart cell culture flask as claimed in claim 7, wherein the detection module is located on an inward side of the bottle body.

9. The smart cell culture flask as claimed in claim 8, wherein the positioning unit is a Global Positioning System chip.
